(19) United States
(12) Patent Application Publication
(10) Pub. No.: US 2017/0314148 A1
(43) Pub. Date: Nov. 2, 2017

(54) ELECTROCHEMICAL CATALYST FOR CONVERSION OF CO2 TO ETHANOL

(71) Applicant: UT-Battelle, LLC, Oak Ridge, TN (US)

(72) Inventors: Adam J. Rondinone, Knoxville, TN (US); Peter V. Bonnesen, Knoxville, TN (US); Dale K. Hensley, Kingston, TN (US); Rui Peng, Oak Ridge, TN (US); Yang Song, Oak Ridge, TN (US)

(21) Appl. No.: 15/143,651

(22) Filed: May 2, 2016

(51) Int. Cl. C25B 11/04; C07C 29/15

(52) U.S. Cl. CPC C25B 11/0447; C25B 11/0415; C07C 29/15; C25B 11/0405

(57) ABSTRACT

The invention provides an electrocatalyst. The electrocatalyst comprises carbon nanospikes (CNS) and copper nanoparticles. The copper nanoparticles are supported on and/or embedded in the CNS. The electrocatalyst can be used to convert carbon dioxide into ethanol.

### STATEMENT REGARDING FEDERALLY SPONSORED RESEARCH

The United States Government has rights in this invention pursuant to contract no. DE-AC05-000R22725 between the United States Department of Energy and UT-Battelle, LLC.

### FIELD OF THE INVENTION

This invention relates to the field of electrocatalysis. In particular, the invention relates to the use of an electrocatalyst for converting carbon dioxide to ethanol.

### BACKGROUND OF THE INVENTION

A low cost, easily implemented and widely distributable means to mitigate or eliminate CO₂ emissions will be necessary to meaningfully address climate change. Closing the carbon cycle by utilizing CO₂ as a feedstock for currently used commodities, in order to displace a fossil feedstock, is an appropriate intermediate step towards a carbon-free future. Direct electrochemical conversion of CO₂ to liquid hydrocarbon fuels could provide a means to close the carbon cycle, and to store and transport energy in a manner appropriate for the existing internal combustion vehicle fleet. Metal-based catalysts, such as copper, platinum, iron, silver and gold have been investigated for CO₂ reduction with some very high Faradaic efficiencies reported for methane conversion. However, efficient electrocatalysts for reducing CO₂ into a desirable liquid fuel are not available. Copper is a metal catalyst for electrochemical CO₂ reduction, capable of reducing CO₂ into more than 30 different products, including carbon monoxide (CO), formic acid (HCOOH), methane (CH₄) and ethane (C₂H₄), but efficiency and selectivity for liquid fuel are too low for practical use. Competing reactions limit the yield of any one liquid product to single-digit percentages.

Polycrystalline Cu foil produces a mixture of compounds in CO₂-saturated aqueous solutions that are dominated either by H₂ at low overpotential, or by CO and HCO₂⁻ at high overpotential, or by hydrocarbons and multi-carbon oxygenates at the most extreme potentials. Theoretical studies predict that graphene-supported Cu nanoparticles would enhance catalytic activity due to the strong Cu-graphene interaction via defective sites, which would stabilize the intermediates from CO₂ reduction and improve selectivity towards hydrocarbon products as methane and methanol at lowered overpotential. Early studies revealed that the electrode surface was dominated by adsorbed CO during the CO₂ reduction and that CO acted as intermediate in the production of hydrocarbons. Cu produces hydrocarbons and multi-carbon oxygenates when supplied with CO in the absence of CO₂, but very negative potentials are still required to promote CO reduction over H₂ evolution. Large overpotentials preclude energetically efficient electrolysis and favor hydrocarbons over liquid oxygenates. Recently, high selectivity of CO electroreduction to oxygenates, with ethanol as the major product, was achieved by oxide-derived Cu, in which the surface intermediates were stabilized by the grain boundaries. The next milestone is the full reduction to liquid fuel, directly from CO₂.

### SUMMARY OF THE INVENTION

In one aspect, the present invention provides an electrocatalyst. The electrocatalyst comprises carbon nanospikes (CNS) and copper nanoparticles. The copper nanoparticles are supported on and/or embedded in the CNS.

In another aspect, the invention provides a method of converting carbon dioxide into ethanol. The method comprises contacting an electrocatalyst comprising carbon nanospikes (CNS) and copper nanoparticles supported on and/or embedded in the CNS with carbon dioxide, and applying a voltage thereto to covert the carbon dioxide into ethanol.

### BRIEF DESCRIPTION OF THE FIGURES

FIG. 1. High-resolution transmission electron microscopy electrodeposited copper nanoparticles on CNS electrode. Electrodeposited particles are imbedded in N-doped CNS providing intimate contact between copper surface and alpha-carbon reactive sites.

FIG. 2. Linear sweep voltammetry (LSV) curves in potential range of 0.00 to −1.35 V vs. RHE.

FIG. 3. Fractional Faradaic efficiency at various potentials. Up to −0.9 V only gas phase products are produced. At more negative potentials, the rate of CO radical production is high enough to allow for CO dimerization to occur, producing C2 products.

FIG. 4. Partial current density of CO₂ reduction products from the Cu/CNS electrode at various potentials.

FIG. 5. (a) The intermediate species OCH₂CH₃ is chemically adsorbed on N-doped CNS. There are two routes for further electroreduction: (b) the cleavage of the CNS-oxygen bond to produce ethanol; (c) the cleavage of the C—O bond in OCH₂CH₃ to form ethane. According to DFT calculations, the former reduction route is much more energetically favorable (more stable by 1.59 eV), consistent with the experiment observation that ethanol is the only C2 product.

FIG. 6. A hypothetical reaction mechanism that is supported by our first principles calculations is dependent on proximity of multiple reactive sites on the Cu particle and the CNS, which is provided by the nanostructured morphology of the catalyst.

### DETAILED DESCRIPTION OF THE INVENTION

In one aspect, the invention provides an electrocatalyst comprising (i) highly textured nitrogen (N)-doped graphene that portrays a surface of intense folds and spikes, termed carbon nanospikes (CNS); and (ii) copper (Cu) nanoparticles. The CNS in the electrocatalyst can have any length. Generally, the nanospike length may be precisely, about, or at least, for example, 30, 35, 40, 45, 50, 55, 60, 65, 70, 75, 80, 85, or 90 nm, or within a range bounded by any two of these values.

Each nanospike is composed of layers of puckered carbon ending in a curled tip. Typically, the width of the curled tip may be precisely, about, or at least, for example, 0.5, 0.6, 0.7, 0.8, 1.0, 1.1., 1.2, 1.3, 1.4, 1.5, 1.6, 1.7, 1.8, 1.9, 2.0, 2.1, 2.2, 2.3, 2.4, or 2.5 nm, or within a range bounded by any two of these values.

The CNS are doped with nitrogen. The amount of nitrogen in the CNS may be precisely about, or at least, for example, 3, 4, 5, 6, 7, 8, or 9% atm., or within a range bounded by any two of these values.

The N-doped CNS can be prepared by any method known to those skilled in the art. Suitable methods include, for example, those methods described in Sheridan et al., J. of Electrochem. Society, 2014, 161(9): H558-H563, and described in Example 1 below.

The Cu nanoparticles are supported on, and/or, imbedded in the CNS. When the Cu nanoparticles are supported on, and/or, imbedded in the CNS, it enables the Cu nanoparticles and CNS to be in close proximity thus providing intimate contact between the Cu surface and the carbon reactive sites.

The Cu nanoparticles can be any nanosize. Generally, the Cu nanoparticles may be precisely, about, or at least, for example 10, 15, 20, 25, 30, 35, 40, 45, 50, 55, 60, 65, 70, 75, 80, 85, 90, 95, 100, 105, 110, 115, or 120 nm, or within a range bounded by any two of these values. In one embodiment, the Cu nanoparticles can be about 40 nm in size.

The Cu nanoparticles can be present on the CNS at any density. Usually, the density of the Cu nanoparticles on the CNS may be, precisely, about, or at least, for example 0.5×10¹⁰, 0.8×10¹⁰, 0.9×10¹⁰, 1.0×10¹⁰, 1.2×10¹⁰, 1.3×10¹⁰, 1.4×10¹⁰, 1.5×10¹⁰, 1.8×10¹⁰, 2.0×10¹⁰, 2.5×10¹⁰, or 3.0×10¹⁰particles/cm², or within a range bounded by any two of these values. In one embodiment, the Cu nanoparticles are present on the CNS in a density of about 1.2×10 particles/cm².

The coverage of Cu nanoparticle on CNS can be any amount. Generally, the coverage of Cu nanoparticle on CNS is approximately 5, 6, 7, 8, 9, 10, 11, 12, 13, 14, 15, 16, 17, 18, 19, 20, 25, 30, 35, 40, 45, 50, 55, 60, 65, 70, or 75%, or within a range abounded by any two of these values. In one embodiment, the coverage of Cu on CNS is about 14%.

The Cu nanoparticles can be applied to the CNS using any method such that it results in the Cu nanoparticles being supported and/or imbedded in the CNS. Such methods include for example electronucleation. For example, the nanoparticles of Cu can be electronucleated from CuSO₄ directly onto the CNS. Briefly, for instance, a CNS electrode is emerged into an aqueous electrolyte with CuSO₄ and H₂SO₄, which was degassed and then purged by N₂. Voltage in then applied on the CNS electrode to reduce Cu²⁺ to Cu onto the CNS. Variation of electronucleation condition may result in different morphologies in Cu nanoparticles and variate interaction between Cu nanoparticles and CNS.

Further methods include, but are not limited to Physical Vapor Deposition (PVD) and Chemical Vapor Deposition (CVD). Additional methods include thermal decomposition of absorbed Cu-content organometallic complex, and chemical reduction or hydrothermal reduction of absorbed Cu salts, such as, for example, Cu(acetate)₂, CuCl₂ and CuSO₄.

In another aspect, the invention provides a method of converting carbon dioxide into ethanol. The method comprises contacting the Cu/CNS electrocatalyst described above with carbon dioxide, and applying a voltage to covert the carbon dioxide into ethanol. The carbon dioxide and Cu/CNS electrocatalyst can be contacted by any method known to those in the art. For example, the carbon dioxide gas can be pumped across the Cu/CNS electrocatalyst. In another example, the carbon dioxide can be dissolved in water, and flowed over the Cu/CNS electrocatalyst.

Typically any negative voltage can be used in the method of the present invention to convert carbon dioxide to ethanol. Generally, the voltage may be precisely, about, or at least, for example −0.5, −0.7, −0.9, −1.0, −1.2, −1.4, −1.5, −1.7, −2.0, −2.1, −2.5, −2.7, or −3.0, or within a range bounded by any two of these values. In one embodiment, the optimum voltage for ethanol production is approximately −1.2 volts vs a reversible hydrogen electrode.

The voltage can be applied by any method known to those skilled in the art. For example, the voltage can be applied by fixing a wire to the electrode, immersing the electrode in a CO₂-saturated bicarbonate solution, and applying the voltage without regard to current. A counter electrode (platinum or carbon) is used to complete the cell, and a reference electrode is used to control potential.

In one embodiment, the carbon dioxide is converted into deuterated ethanol, CD₃CD₂OD, where D represents deuterium. Deuterated ethanol can be formed by, for example, dissolving the carbon dioxide in heavy water (deuterium oxide, D₂O) instead of water (H₂O), and using deuterated salts such as KDCO₃ in place of KHCO₃, as needed, in the electrolyte.

The Cu/CNS electrocatalyst of the present invention exhibits much higher selectivity for CO₂ electroreduction than H₂ evolution, with a subsequent high Faradaic efficiency to produce ethanol. Without wishing to be bound by theory, this results both from an increase in the intrinsic CO₂ reduction activity of Cu and from the synergistic interaction between Cu and neighboring N-doped CNS, which controls reduction to alcohol. The major CO₂ reduction product is ethanol, which corresponds to a 12 e⁻ reduction with H₂O as the H⁺ source, where E is the equilibrium potential. The total reaction is:

2 CO₂+9 H₂O+12 e⁻→C₂H₅OH+12 OH⁻ E⁰=0.084 V vs. SHE

By comparing Cu/CNS to control electrodes comprised of Cu/C-Film (glassy carbon) and bare CNS, CO₂ reduction activity is not a simple consequence of either Cu or CNS. Rather, CO₂ reduction involves the interaction between adjacent catalytic sites on the Cu and CNS, facilitated by the nanostructured morphology of the electrocatalyst.

Examples have been set forth below for the purpose of illustration and to describe certain specific embodiments of the invention. However, the scope of this invention is not to be in any way limited by the examples set forth herein.

### EXAMPLES

### Example 1

### Characterization of Carbon Nanospike Electrode

The bare CNS electrode was characterized as a dense nanotextured carbon film terminated by randomly oriented nanospikes approximately 50-80 nm in length, where each nanospike consists of layers of puckered carbon ending in a ˜2 nm wide curled tip. The film is grown by a plasma-enhanced chemical vapor deposition reaction using acetylene and ammonia as reagents. Raman spectra indicated that CNS have similar structure to disordered, multilayer graphene. XPS indicated nitrogen doping density as 5.1±0.2% atm, with proportions of pyridinic, pyrrolic (or piperidinic) and graphitic nitrogens of 26, 25 and 37% respectively, with the balance being oxidized N.

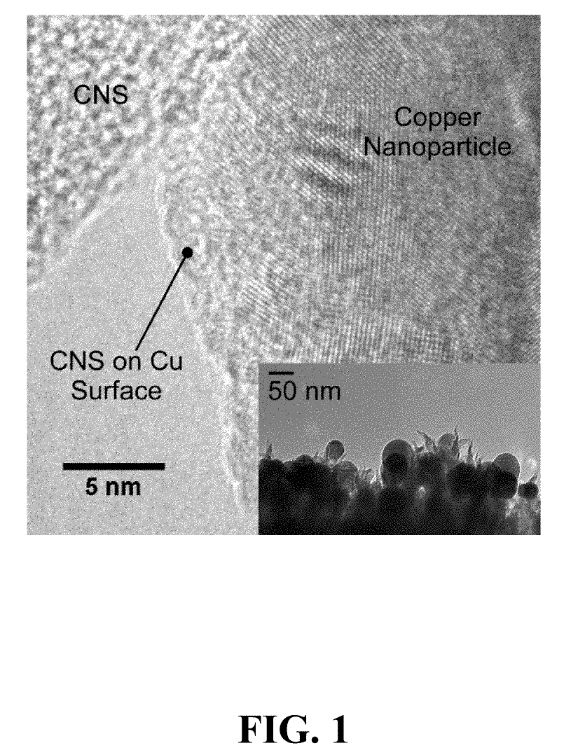

In the current experiment, nanoparticles of Cu were electronucleated from CuSO₄ directly onto the CNS, and imaged via SEM. These well-dispersed Cu particle sizes ranged from about 30 nm to 100 nm with average size of 39 nm, with a density ca. 1.2×10¹⁰ particles cm⁻². According to the average particle size, the coverage of Cu on CNS is ca. 14.2%. TEM measurements (FIG. 1 inset) confirm particle size observed via SEM. High-resolution transmission electron microscopy on scraped samples (HR-TEM) illustrates the Cu/CNS interface (FIG. 1) and illustrate a close proximity between Cu and CNS. The lattice spacing of this representative Cu nanoparticle was measured as 0.204 nm, which is consistent with Cu. Cu₂O with lattice spacing ca. 0.235 nm were present on Cu nanoparticles surface, likely resulting from exposure to air during sample preparation and transportation between measurements. Electronic Energy Loss Spectroscopy (EELS) measurements indicate a graphitic carbon, and confirm the CNS wrapped around the Cu nanoparticles (FIG. 1).

### Example 2

### Stability of Cu/CNS Catalyst

To investigate the short-term stability of the Cu/CNS catalyst, additional HR-TEM images and EELS spectra were taken after a 6-hour CO₂ reduction reaction, and no obvious changes were observed. Likewise, X-ray Photoelectric Spectroscopy (XPS) measurements for Cu 2p_{3/2} showed a similar asymmetric peak at 932 eV, indicating that the Cu nanoparticles were stable after a 6 hour reaction and were mainly comprised of Cu⁰. However, after a 6-hour electroreduction the fraction of graphitic-N significantly decreased (38.9 to 10.7%), while pyridinic-N and pyrrolic/amine-N increased (14.2 to 24.7% and 39.6 to 54.2%, respectively. While XPS cannot distinguish between pyrrole and amine, electroreduction from pyridinic-N to pyrrolic-N would require removal of a C atom, therefore the increased pyrrolic/amine-N is likely piperidine, with no increase in pyrrolic fraction. No change in electrochemical activity was observed during this prolonged electroreduction.

### Example 3

### Electroreduction Activity

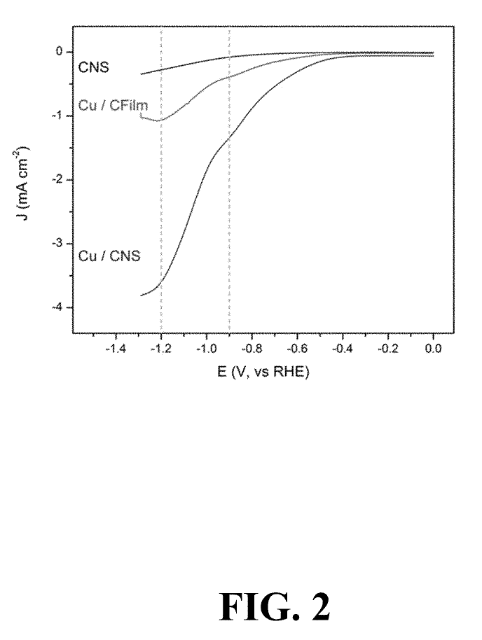

CO₂ electroreduction activity was first measured by linear sweep voltammetry (LSV) in potential range −0.00 to −1.30 V vs. RHE in the presence of CO₂ saturated electrolyte as shown in FIG. 2. Larger current densities were obtained in Cu/CNS than either Cu/C-Film or bare CNS electrodes, and the Cu/CNS onset potential for CO₂ reduction was −0.3 V more positive than CNS without Cu particles. Note that two well-defined reduction waves appeared at −0.9 V and −1.20 V vs. RHE in Cu/CNS LSV curves.

To investigate the mechanism of the electrochemical reaction, 60-minute chronoamperometry (CA) measurements conducted over a potential range from −0.7 to −1.3 V, which included these two reduction waves, were carried out. New electrodes were fabricated for each data point. The gaseous and liquid products of each CA run were analyzed by gas chromatography (GC) and NMR (of headspace and electrolyte, respectively) to calculate overall current density and Faradaic efficiency for CO₂ reduction and for each product. The overall sustained current density for CO₂ reduction, J_{CO2 redn} was increased with more negative potential in all three electrodes consistent with that shown in LSV curves. Cu/CNS electrode had greater propensity for CO₂ reduction than either Cu/C-Film and bare CNS electrodes, for instance, J_{CO2 redn} from Cu/CNS was 5-fold higher than for bare CNS and 3-fold higher than for Cu/C-Film, at −1.2 V.

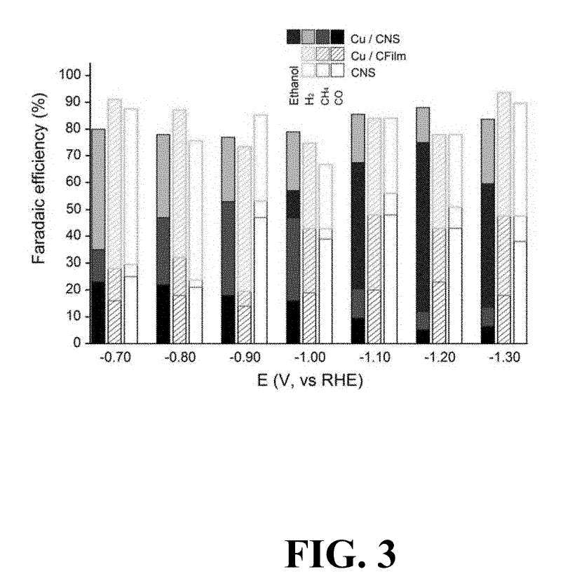

The fractional Faradaic efficiency was computed by dividing the total electrons into each product (determined independently by chemical analysis) by the total electrons passed during the amperometry experiment. Due to experimental losses between the anode and cathode, the total fractions are less than 100%. The fractional Faradaic efficiency is shown in FIG. 3.

At −0.9 V vs. RHE and more positive potential, only gas phase products H₂, CO and CH₄ were obtained from all three electrodes. At −1.0 V vs. RHE and more negative potential, ethanol is produced as a liquid, soluble in the aqueous electrolyte. Trace formic acid is occasionally detected by NMR. Remarkably, ethanol is the only liquid phase product from Cu/CNS, and is not detectable from Cu/C-Film and bare CNS control electrodes. Ethanol, as a C2 product, requires carbon-carbon coupling at some point during the reduction reaction. In comparison, neither control electrode produced C2 products, only C1 products CO and CH₄. Efforts were made to observe other products more commonly produced by copper electroreduction, such as methanol, ethane or ethylene but none were detected by either GC or NMR.

Examining the breakdown of Faradaic efficiencies for various reactions on Cu/CNS, reveals that at −1.2 V, ethanol conversion exhibited the highest efficiency at 63% (that is, 63% of the electrons passing through the electrode were stored as ethanol). Also at −1.2 V vs. RHE, the Faradaic efficiency of gas phase products methane and CO dropped to 6.8% and 5.2%, respectively. The Faradaic efficiency of CO₂ reduction (competing against water reduction) is 75%. This means that under the best conditions, the overall selectivity of the reduction mechanism for conversion of CO₂ to ethanol is 84%.

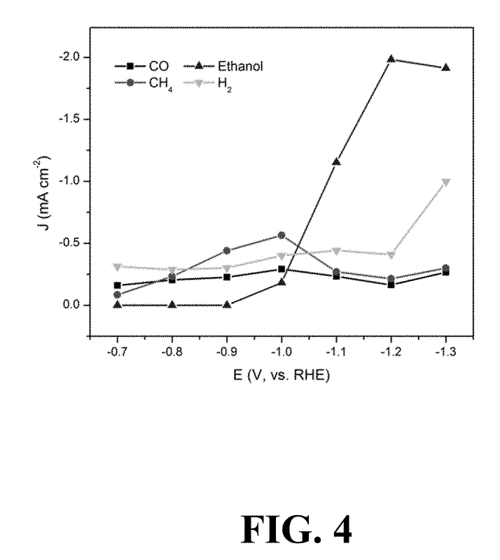

The fraction current density for each product exhibited volcanic shape dependence to the potentials applied on the Cu/CNS, as illustrated in FIG. 4. The maximum current density for methane was observed at −1.0 V vs. RHE, and decreased when ethanol generation began. Then the current for ethanol generation increased with more negative potential until reaching a summit at −1.2 V vs. RHE, where Cu/CNS attained the highest overall CO₂ reduction efficiency. At more negative potential, current density for ethanol and other products from CO₂ reduction remained comparable, however, the Faradaic efficiency value of CO₂ to ethanol conversion declined while the value for H₂ evolution increased significantly. The decline of Faradaic efficiency was the result of the catalysts reaching the mass-transport-limited current density for CO₂ reduction and therefore hydrogen evolution via H₂O reduction at unoccupied active sites.

### Discussion

Previous reports of CO₂ electroreduction on copper have demonstrated a variety of C1 and C2 products, including CO, CH₄, CH₂O₂, ethane, ethylene, ethanol. Heavier hydrocarbons have not been reported. C2 products are hypothesized to form through coupling of CO radicals on the surface of the copper, and a high percentage output of C2 products would indicate a rapid coupling of Cu-bound C1 intermediates, or possibly an electron transfer process that is coupled to C—C bond formation between surface-bound C1 intermediates species and a nearby CO in solution. Ordinarily, on bulk copper the coupled C2 would continue to be reduced to ethane or ethylene so long as the product was in contact with the copper electrode. In contrast, with this experiment we have not been able to detect any C2 product except ethanol, indicating that a reaction mechanism dominates that precludes further reduction to ethane.

The hypothesis is that three electrochemically active species are present in Cu/CNS catalysts: (i) Cu nanoparticles, (ii) the various nitrogen dopants present in the CNS, and (iii) partially positive-charged carbon atoms immediately adjacent to the CNS nitrogen dopants (termed alpha-C). It is predicted that there is a strong interaction between Cu nanoparticle and carbon, and it is expected to extend to CNS as well. The strong interaction provides an environment in which a reaction mechanism involving reactive sites on the Cu surface and on the N-doped CNS may dominate. In this environment, the close proximity and strong interactions promote transfer of intermediate C2 species from the Cu surface to the N-doped CNS. Although we were not able to measure the exact distance between Cu nanoparticle and carbon nanospike, the contact should be direct and intimate according to HR-TEM images.

This transfer is important because the electronic structure near the Fermi level of graphene is modified in N-doped CNS, where localized 7C electronic states are reported to form at the neighboring carbon atoms, and propagate anisotropically around the defect due to the perturbation of the π-conjugated system. Due to electron-withdrawing effects in the graphene π-conjugated system, the alpha-C atoms adjacent to nitrogen are positively polarized. This polarization provides an active site for the C2 intermediates to adsorb.

Concerning the reaction mechanism, following electron transfer to Cu-adsorbed CO₂ to form CO₂^{•-}_{ads}, this anionic radical is reduced to CO_{ads} or other C1 intermediates (CHO_{ads} or CH₂O_{ads}) on the Cu surface:

CO₂+e⁻→CO^{•-}_{2 ads}

CO^{•-}_{2 ads}+e⁻+H₂O→CO_{ads}+2OH⁻

CO_{ads}+e⁻+H₂O→CHO_{ads}+OH⁻

CHO_{ads}+e⁻+H₂O→CH₂O_{ads}+OH⁻

CO and methane will result from further electron transfer to these surface species, whereas C—C coupling may occur among two surface adsorbed intermediates or between a surface species and a CO from solution. At −1.2 V vs RHE, the major product is C2 indicating that at a high enough rate of production of CO radical, C2 coupling is the dominant outcome.

2 CO_{ads}→O*C*CO

CO_{ads}+CHO_{ads}→0*C*CHO

2 CHO_{ads}→*OCHCHO*

CHO_{ads}+CH₂O_{ads}→*OCH₂CHO

2 CH₂O_{ads}→*OCH₂CH₂O

CO_{ads}+CO→O*CCO

Once coupled C2 products are formed, they reduce only to ethanol. In order for ethanol to be the only C2 product, a mechanism must be available that limits the electroreduction to prevent the formation of ethane.

To confirm whether nitrogen dopants and the neighboring alpha-C atoms in the CNS can effectively adsorb the C2 intermediates, first-principles density functional theory (DFT) calculations were carried out. As CNS have similar structure to multilayer graphene, a graphene sheet is adopted to model the interaction between CNS and the C2 intermediates (such as OCCO) for simplicity without losing the essence of the physics. For a pristine graphene sheet, our calculations suggest the binding energy between OCCO and graphene is 0.19 eV with a separation distance −2.95 A (Supplemental FIG. S7a). Interestingly, for N-doped graphene, the N dopant and adjacent alpha-C atoms become indeed more active so that the binding energy with OCCO is increased to 0.64 eV with the separation distance shortened to −2.70 A (Supplemental FIG. S7b). The tripling of the binding energy to 0.64 eV clearly indicates that the C2 intermediates can be adsorbed by N-doped CNS fairly strongly and may not desorb easily at room temperature. Furthermore, it is important to note that CNS are puckered and curled, indicating local corrugation on the surface. It has been shown previously that local deformation or buckling could enhance the molecular adsorption on carbon nanotubes and graphene. The buckling of pristine and N-doped graphene were considered to investigate the local curvature effect on OCCO adsorption. Upon buckling, the binding energy between OCCO and the concave of pristine graphene is increased to 0.34 eV, while the binding energy between OCCO and the concave of N-doped graphene is enhanced to 0.74 eV. Therefore, the corrugation and curvature naturally embedded into CNS appear to strengthen the binding between CNS and the C2 intermediates.

Consequently, we expect that the nearby N-dopant and alpha-C in the CNS, which is in intimate contact with the Cu surface, adsorbs one of the C2 carbonyls. Further electroreduction then occurs preferentially on the other C2 carbonyl at the Cu surface:

CNS . . . OCCO+5e⁻+5H⁺→CNS . . . OCH₂CH₃

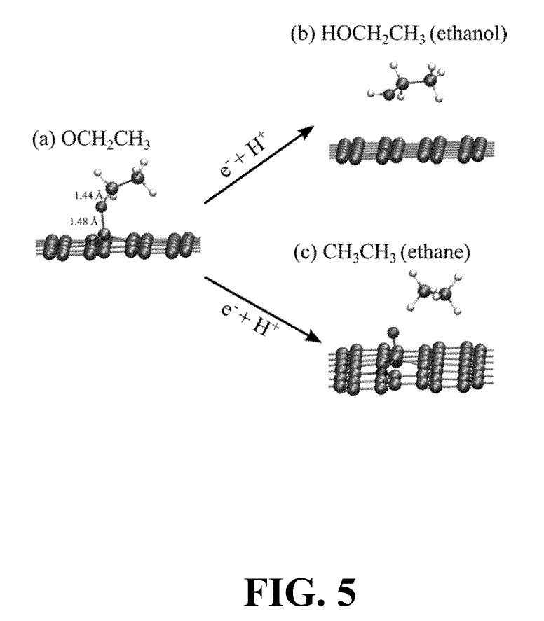

At this stage, the two carbon atoms in the intermediate species OCH₂CH₃ are saturated, while the oxygen atom becomes non-saturated. As a result, calculations show that the CNS-oxygen bond changes from fairly strong physisorption to much stronger chemisorption, and the separation distance is reduced to 1.48 Å (FIG. 5a). As discuss previously, XPS indicates that some graphitic-N is electrochemically reduced to piperidinic-N during a prolonged electroreduction experiment. According to calculations, the binding energy between OCCO and piperidinic-N doped graphene is −0.62 eV, similar to that between OCCO and graphitic-N doped graphene (˜0.64 eV). Therefore the reaction mechanism should occur similarly between both sites. Now there are two routes for further reduction: the cleavage of the CNS-oxygen bond to produce ethanol (FIG. 5b); or the cleavage of the C—O bond in OCH₂CH₃ to form ethane (FIG. 5c). The former reduction route is much more energetically favorable (more stable by 1.59 eV), consistent with the experiment observation that ethanol is the only C2 product. Hence we expect that further reduction cleaves the CNS-oxygen bond on the first carbonyl, producing ethanol.

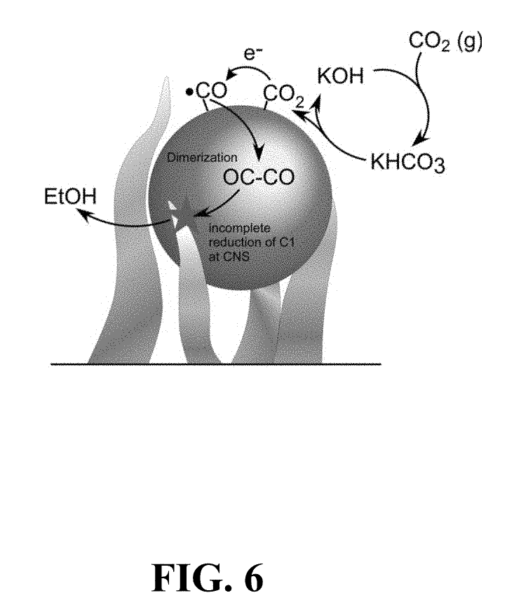

An illustration of the overall process is presented in FIG. 6. In this mechanism, the novel functionality is due primarily to the proximity of multiple reactive sites, which is in turn due to the nanostructured morphology of the catalyst. This demonstrates an important concept, that the selectivity of a reaction can be tuned solely based on morphology and distance between reactive sites. The change in product output with varying potential also yields some insight into the mechanism. At low potentials, alcohol is not produced nor is any C2 product. This is likely due to the rate limiting step being the first reduction of CO₂ on the Cu surface. At higher overpotential, the concentration of reduced CO species on the Cu surface is increased, yielding a greater likelihood of C2 coupling and subsequent ethanol production. At lower concentrations of CO species, no coupling occurs and the product partially reduces to CO or fully reduces to methane. This reaction mechanism is supported by in situ, electrochemical Raman measurements. Without applied potential only a CO₃²⁻ stretching band at 1020 cm⁻¹ was observed on Cu/CNS (in addition to the broad G band at 1610 cm⁻¹ and D band at 1370 cm⁻¹ from multilayer graphene in CNS substrate). It may be adsorbed CO₃²⁻ on CNS or bicarbonate in bulk electrolyte. Immediately when a negative potential was applied the peaks at 1460 and 1520 cm⁻¹ arose, indicating surface intermediates were being generated. These peaks could be assigned to C—H stretching and CH₃ deformation, respectively, in agreement with the electrochemical experiments. At −1.2 V or more negative potential, a new peak arose at 1070 cm^{'1} that is assigned to alkoxyl or alcohol. This peak appeared immediately as potential was applied and disappeared when potential was removed, hence it's concluded that it primarily represents surface adsorbed species rather than products diffused into electrolyte. Considering ethanol was the only detectable product in solution, the peak at 1070 cm⁻¹ is assigned to ethoxyl C—O stretching in ethanol or its intermediate precursor.

This catalyst operates at room temperature and in water, and may be turned on and off easily. Other catalytic processes have been optimized over the past century to reduce CO₂ to alkanes, methanol or higher alcohols. Although many of these prior processes are efficient, they all require high temperatures and pressures (typically 250-400° C. and 50-150 atm.) that are poorly matched to utilization of diffuse renewable energy sources. Electrolytic syntheses enabled by the catalysis with Cu/CNS could provide a more direct, rapidly switchable and easily implemented route to distributed liquid fuel production powered by variable renewable energy sources, such as wind and solar.

What is claimed is:

1. An electrocatalyst comprising carbon nanospikes and copper nanoparticles, wherein the copper nanoparticles are supported on and/or embedded in the carbon nanopsikes.

2. The electrocatalyst according to claim 1, wherein the carbon nanospikes are doped with nitrogen.

3. The electrocatalyst according to claim 1, wherein the carbon nanospikes comprises layers of puckered carbon.

4. The electrocatalyst according to claim 3, wherein the carbon nanospike contains a curled tip.

5. A method of converting carbon dioxide into ethanol comprising:
(i) contacting the electrocatalyst of claim 1 with carbon dioxide, and
(ii) applying a voltage thereto to covert the carbon dioxide into ethanol.

6. A method according to claim 5, wherein the ethanol is deuterated ethanol.

7. A method according to claim 4, wherein the voltage is approximately −1.2 volts.
